(19) United States
(12) Patent Application Publication
(10) Pub. No.: US 2025/0366907 A1
(43) Pub. Date: Dec. 4, 2025

(54) CATHETER ASSEMBLIES AND SYSTEMS WITH INTERLOCKING COMPONENTS AND METHODS FOR FORMING FISTULAS

(71) Applicant: TVA Medical, Inc., Franklin Lakes, NJ (US)

(72) Inventors: Kristin N. Rominger, Franklin Lakes, NJ (US); Alex Palmer, Franklin Lakes, NJ (US); Andy Moll, Franklin Lakes, NJ (US); Breanna Simpson, Franklin Lakes, NJ (US); Olivia R. Palmer, Franklin Lakes, NJ (US); Oladipo Peter Akerele-ale, Franklin Lakes, NJ (US)

(21) Appl. No.: 18/867,407

(22) PCT Filed: May 19, 2022
(86) PCT No.: PCT/US2022/030030

§ 371 Date: Nov. 19, 2024

(51) Int. Cl. A61B 18/14; A61B 18/00; A61M 25/01

(52) U.S. Cl. CPC A61B 18/1492; A61M 25/0127; A61B 2018/00172; A61B 2018/00404; A61B 2018/144

(57) ABSTRACT

A catheter includes a catheter body and a modification device. The catheter further includes a nesting region. The modification device of the catheter is configured to project from a working site of the catheter. The nesting region of the catheter is configured to geometrically engage a nesting region of a second catheter.

### TECHNICAL FIELD

The present disclosure relates to assemblies, systems, and methods for forming a fistula, and more particularly assemblies, systems, and methods with interlocking components for increasing coaptation between catheters for fistula formation.

### BACKGROUND

A fistula is generally a passageway formed between two internal organs. Forming a fistula between two blood vessels can have one or more beneficial functions. For example, the formation of a fistula between an artery and a vein may provide access to the vasculature for hemodialysis patients. Specifically, forming a fistula between an artery and a vein allows blood to flow quickly between the vessels while bypassing the capillaries. In other instances, a fistula may be formed between two veins to form a veno-venous fistula. Generally, fistula formation requires surgical dissection of a target vein, and transecting and moving the vein for surgical anastomosis to the artery. It may therefore be useful to find less invasive and reliable devices and methods for forming a fistula between two blood vessels.

### SUMMARY

One challenging aspect of forming a fistula between blood vessels, though other body vessels are contemplated and possible, is properly aligning and coapting catheters in adjacent blood vessels prior to fistula formation. Accordingly, a need exists for alternative systems, methods, and catheters for fistula formation that ensure catheter alignment and coaptation. Embodiments of the present disclosure are directed to improvements over the above limitations by providing catheter assemblies including geometrically engaging nesting regions.

In one embodiment, a catheter includes a catheter body, a modification device, and a nesting region. The modification device is configured to project from a working site of the catheter. The nesting region is configured to geometrically engage a nesting region of a second catheter.

In another embodiment, a system for forming a fistula between two blood vessels includes a first catheter comprising a first catheter body and a tongue. The tongue includes a tongue body and a tongue projection that extends from the tongue body. The system further includes a second catheter comprising a second catheter body and a groove. The groove comprises a groove body and a groove recess in the groove body. The groove recess is configured to receive the tongue projection. At least one of the first catheter or the second catheter comprises a modification device.

In another embodiment, a method of forming a fistula between a first blood vessel and a second blood vessel includes advancing a first catheter into the first blood vessel. The first catheter includes a first catheter body, a first working site, and a tongue. The tongue includes a tongue body and a tongue projection that extends from the tongue body. The method also includes advancing a second catheter into the second blood vessel adjacent to the first blood vessel. The second catheter includes a second catheter body, a second working site, and a groove. The groove includes a groove body and a groove recess in the groove body. The groove recess is configured to receive the tongue projection. The method also includes forming the fistula between the first blood vessel and the second blood vessel at the first working site and the second working site.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts a side view of a catheter, according to one or more embodiments shown and described herein;

FIG. 2 schematically depicts a side view of a catheter, according to one or more embodiments shown and described herein;

FIG. 3 schematically depicts a longitudinal cross-sectional view of the catheter of FIG. 1, according to one or more embodiments shown and described herein;

FIG. 4 schematically depicts a longitudinal cross-sectional view of the catheter of FIG. 2, according to one or more embodiments shown and described herein;

FIG. 5 schematically depicts a two catheter system, according to one or more embodiments shown and described herein;

FIG. 6 schematically depicts a two catheter system, according to one or more embodiments shown and described herein;

FIG. 7 schematically depicts a side view of a catheter including a transition region, according to one or more embodiments shown and described herein;

FIG. 8 schematically depicts a side view of a catheter including a transition region, according to one or more embodiments shown and described herein;

FIG. 9 schematically depicts a two catheter system, according to one or more embodiments shown and described herein;

FIG. 10 schematically depicts a longitudinal cross-sectional view of a nesting region with a tongue body in a low-profile configuration, according to one or more embodiments shown and described herein;

FIG. 11 schematically depicts a longitudinal cross-sectional view of the nesting region of FIG. 10 with the tongue body in an extended configuration, according to one or more embodiments shown and described herein;

FIG. 12 schematically depicts a two catheter system, according to one or more embodiments shown and described herein;

FIG. 13 schematically depicts a two catheter system, according to one or more embodiments shown and described herein;

FIG. 14 schematically depicts a side view of a catheter, according to one or more embodiments shown and described herein;

FIG. 15 schematically depicts nesting regions, according to one or more embodiments shown and described herein;

FIG. 16 schematically depicts nesting regions, according to one or more embodiments shown and described herein;

FIG. 17 schematically depicts nesting regions, according to one or more embodiments shown and described herein;

FIG. 18 schematically depicts nesting regions, according to one or more embodiments shown and described herein; and

FIG. 19 schematically depicts nesting regions, according to one or more embodiments shown and described herein.

### DETAILED DESCRIPTION

Embodiments described herein are directed to devices and methods for forming a fistula. In some embodiments, the devices and methods may be used to form a fistula between two blood vessels. More particularly, a catheter may be placed in each of two adjacent blood vessels to form a fistula therebetween with the catheters.

Fistula forming elements may be mounted to catheters which may then be used to form a fistula between vessels. However, flexibility of the catheters, spacing of vessels, thickness of the vessel walls, and/or the tortuous anatomy of the vessels, may make it difficult to provide sufficient coaptation and/or alignment between vessels for fistula formation. The embodiments described herein address the one or more aforementioned limitations. In particular, the devices and methods for forming a fistula described herein may include a first catheter, having a catheter body, a modification device, such as an electrode, and a nesting region. The modification device is configured to project from a working site of the catheter and modify a blood vessel wall. The nesting region is configured to geometrically engage a nesting region of a second catheter having a corresponding geometry. When geometrically engaged, the nesting regions of the catheters may interlock. The nesting regions may further be able to magnetically mate to enhance geometric engagement. When geometrically engaged, the nesting regions promote alignment and coaptation between the first and second catheters in adjacent blood vessels. Particularly, geometric engagement of the nesting regions may promote the alignment and coaptation of the working site of the first catheter and a working site of the second catheter before and during fistula formation. Various embodiments will now be described in greater detail below with reference to the figures.

As used herein, the term “proximal” means closer to or in the direction of an origin of an element, such as a catheter. The origin of a catheter may be a handle or other user-manipulated portion of the catheter. The term “distal” means further from the origin, or handle, of the catheter. Put another way, the term “distal” means closer to or in the direction of a tip of a catheter, which is separated from a handle of the catheter by the length of the catheter body.

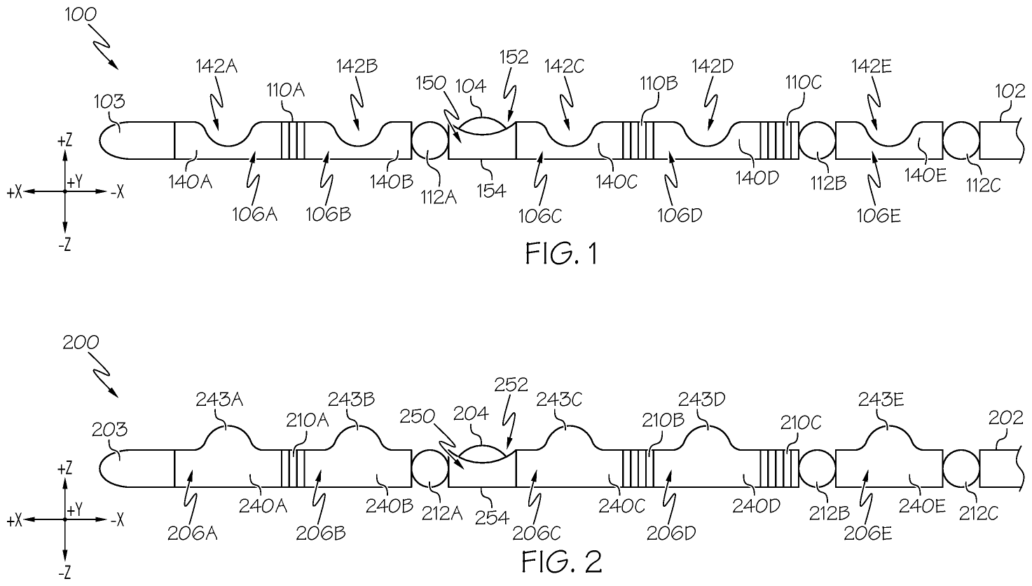

Referring now to FIG. 1, a catheter 100 (e.g., a first catheter) of a system for forming a fistula is depicted. As will be described in greater detail herein, the system may further include a second catheter 200 (FIGS. 2 and 4). While the structure of the catheter 100 will be discussed in detail, it should be appreciated that the structure of the second catheter 200 (FIGS. 2 and 4) may mirror the catheter 100 except where noted. Still referring to FIG. 1, the catheter 100 generally includes a catheter body 102, which may include a distal tip 103 that is particularly configured to aid in advancement of the catheter 100 through a blood vessel. For example, the distal tip 103 may be pointed and/or atraumatic for advancement through a blood vessel. The catheter body 102 may have any desirable cross-sectional shape and any suitable diameter for intravascular use. The catheter 100 may further include one or more lumens or other passageways extending at least partially along or through the catheter body 102. For instance, the one or more lumens may extend at least partially longitudinally through the catheter body 102 in the direction of the x-axis of the coordinate axes of FIG. 1.

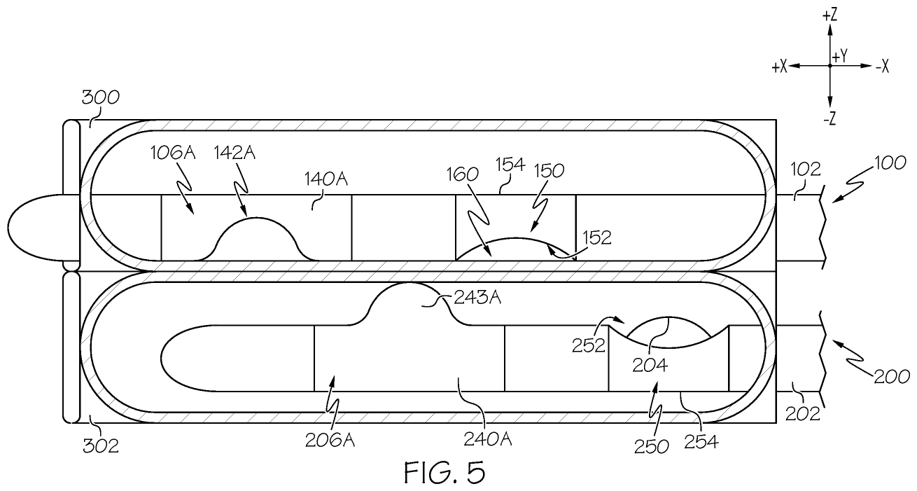

The catheter 100 may further include a working site 150 arranged along the catheter body 102. The working site 150, as described herein, refers to a portion of the catheter 100 positioned along the catheter body 102 that is configured to modify a blood vessel (e.g., cut, ablate, etc.). In particular, in embodiments of the present disclosure, the catheter 100 may include a working site 150 that is configured to form one or more fistulas between a first blood vessel 300 (FIG. 5) and a second blood vessel 302 (FIG. 5). In embodiments, the working site 150 may be positioned along the catheter body 102 at a position proximal to (e.g. in the −x direction of the coordinate axes of FIG. 1) the distal tip 103 and define an active side 152 of the catheter 100. The working site 150 may comprise one or more openings in the active side 152 of the working site 150 that allow for passage of one or more instruments into and/or out of the catheter body 102 for modifying the vessel. For example, the active side 152 of the working site 150 is the portion of the working site 150 that abuts or faces the area of the vessel where a modification is to be made. For instance, an electrode 104 may protrude from the active side 152 of the working site 150 and extend radially away from a longitudinal centerline of the catheter 100 to contact a wall of the blood vessel. The active side 152 of the catheter 100 extends across the catheter body 102, the working site 150, and any other elements arranged along the catheter body 102.

Embodiments including the electrode 104 are discussed in detail herein. However, it is noted that other cutting or modification devices are contemplated and possible. For instance, ultrasonic cutting elements, laser, knives, etc. may be used in place of or in addition to the electrode 104. “Modification device,” as used herein, generally refers to any cutting or modification device, including the electrode 104, that may be used to modify (e.g. cut, ablate) a vessel wall.

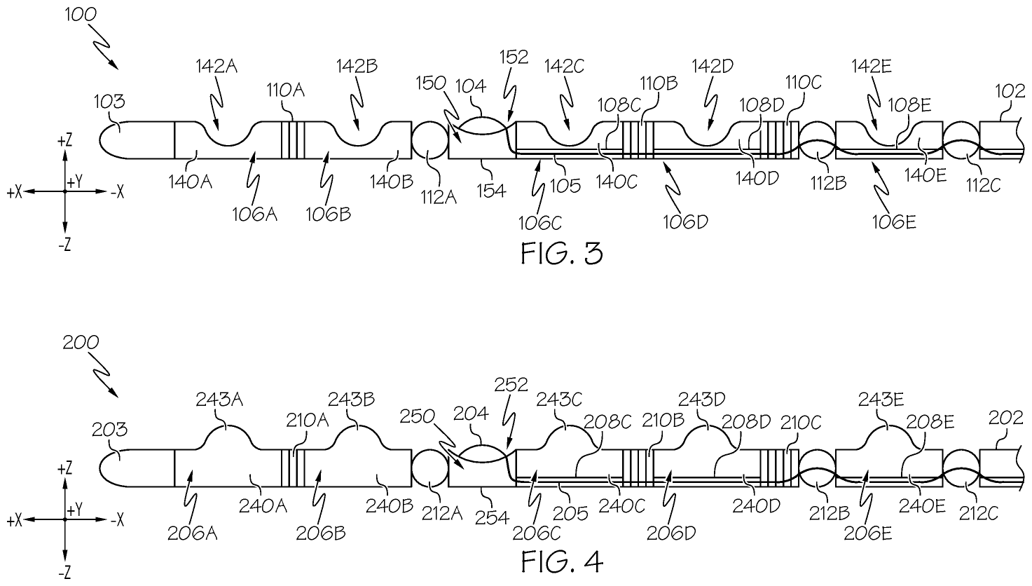

The electrode 104 may include an exposed ablation surface, which may be activated to ablate tissue, and a lead wire, or electrode wire, 105 (FIG. 3) or other conductor attached thereto. Particularly, when activated, current may be supplied to and/or carried from tissue and fluid via the ablation surface to facilitate ablation or vaporization of tissue to form a fistula. In some embodiments, the electrode 104 may be a spring wire or leaf spring electrode, which may be movable between a retracted configuration, in which the electrode 104 is retained within the catheter 100, and a protruding configuration, in which electrode 104 projects from a surface of the catheter body 102. The electrode 104 may or may not be naturally biased to project from the catheter body 102. When the electrode 104 is naturally biased to project from the catheter body 102, a structure, such as a sleeve, may be used to hold or maintain the electrode 104 in a retracted configuration until deployment is desired. In some embodiments, the catheter body 102 may comprise one or more insulating materials (not shown) which may shield or otherwise protect the catheter 100 and its components from heat generated by the electrode 104 during use.

Still referring to FIG. 1, the catheter 100 includes a non-active side 154 or region positioned opposite the active side 152 of the working site 150. For example, the non-active side 154 refers to the side of the catheter 100 devoid of cutting and/or ablation means. The non-active side 154 of the catheter 100 extends across the catheter body 102, the working site 150, and any other elements arranged along the catheter body 102. In other words, the catheter body 102, the working site 150, and any additional elements arranged along the catheter body 102 (e.g. nesting regions 106A-E) may each define the non-active side 154. The non-active side 154 of the catheter 100 is diametrically opposite the active side 152 of the catheter 10. Therefore, the non-active side 154 of the catheter 100 is positioned opposite the electrode 104. In other words, the non-active side 154 of the catheter 100, does not abut or face a modification being formed in the blood vessel 300 (FIG. 5) via the active side 152 of the working site 150. Instead the non-active side 154 of the catheter 100 may be spaced from a modification being formed in the blood vessel 300 (FIG. 5) by the electrode 104 by the diameter or height (e.g. in the direction of the z-axis of the coordinate axes of FIG. 1) of at least a portion of the catheter body 102.

Still referring to FIG. 1, the catheter 100 may include one or more arrays of magnets arranged longitudinally along the catheter body 102. For instance, the catheter 100 may include a first array of magnets 110A that extends longitudinally along the catheter body 102 and is positioned distal (e.g. in the +x direction of the coordinate axes of FIG. 1) to the working site 150. In some embodiments, the first array of magnets 110A may be positioned longitudinally between (e.g. in the direction of the x-axis of the coordinate axes of FIG. 1) the working site 150 and the distal tip 103 of the catheter body 102. The catheter 100 may include a second array of magnets 110B that extends longitudinally along the catheter body 102 and is positioned proximal (e.g. in the −x direction of the coordinate axes of FIG. 1) to the working site 150. In embodiments, the catheter 100 may include a third array of magnets 110C. In embodiments, the catheter 100 may include the first array of magnets 110A, the second array of magnets 110B, and the third array of magnets 110C individually or in any combination. It should be appreciated that while the phrase “array of magnets” is used herein, each of the arrays of magnets 110A, 110B, and 110C may be configured as a single magnet along the catheter body 102.

The arrays of magnets 110A-C described herein may be permanent magnets comprising one or more hard magnetic materials, such as but not limited to alloys of rare earth elements (e.g., samarium-cobalt magnets or neodymium magnets, such as N52 magnets) or alnico. In some variations, the arrays of magnets 110A-C may comprise anisotropic magnets; in other variations, the arrays of magnets 110A-C may comprise isotropic magnetics. In some variations, the arrays of magnets 110A-C may be formed from compressed powder. In some variations, a portion of the arrays of magnets 110A-C (e.g., a permeable backing) may comprise one or more soft magnetic materials, such as but not limited to iron, cobalt, nickel, or ferrite. It should be appreciated that in systems comprising two catheters, either the first catheter 100 or the second catheter 200 (FIGS. 2 and 4) may comprise ferromagnetic elements (i.e., elements attracted to but not generating a permanent magnetic field). For example, in some variations, the first catheter 100 may include only one or more ferromagnetic elements while the second catheter 200 (FIGS. 2 and 4) may comprise one or more permanent magnets. In other variations, the second catheter 200 (FIGS. 2 and 4) may include only one or more ferromagnetic elements while the first catheter 100 may comprise one or more permanent magnets. However, in other variations, one or both of the first catheter 100 and the second catheter 200 (FIGS. 2 and 4) may include any suitable combination of ferromagnetic, permanent, and/or other suitable kinds of magnets.

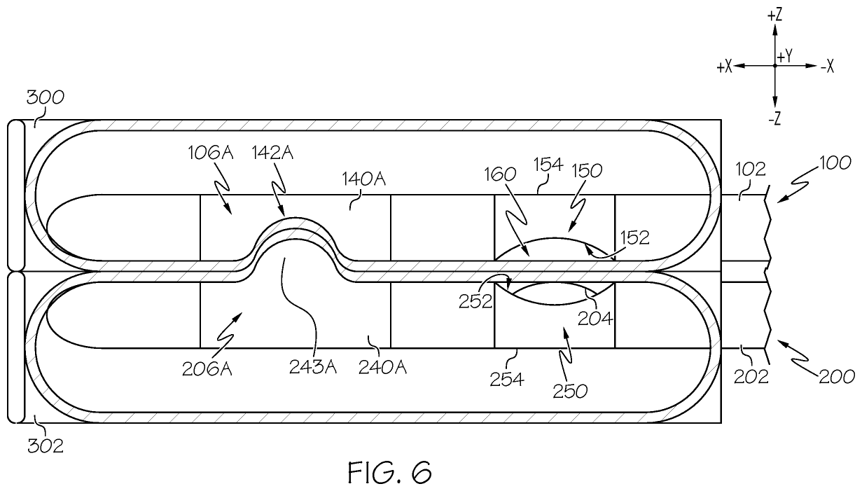

Generally, the dimensions of the arrays of magnets 110A-C described herein may be selected based upon the size of the catheter 100 carrying the arrays of magnets 110A-C, which in turn may be selected based upon the anatomical dimensions of the selected blood vessels through which the catheter 100 may be advanced. For example, if the catheter 100 is to be advanced through a blood vessel 300 (FIGS. 5 and 6) having an internal diameter of about 3 mm, it may be desirable to configure any array of magnets 110A-C to be less than about 3 mm at the widest part of their cross-sections, to reduce the risk of unintended contact with the vessel walls during advancement and manipulation of the catheter 100. Each array of magnets 110A-C may have any suitable length (e.g., about 5 mm, about 10 mm, about 15 mm, about 20 mm, and the like), although it should be appreciated that in some instances longer arrays of magnets may limit the flexibility of the catheter 100 to maneuver through a vessel. In some variations, the arrays of magnets 110A-C may include a plurality of cuboid magnets. In other embodiments, each magnet of the arrays of magnets 110A-C may have any suitable shape for placement inside or on the catheter. Magnets may be cylindrical, semi-cylindrical, tube-shaped, box-shaped, or the like.

In embodiments, the outer surfaces of the arrays of magnets 110A-C may be flush or in line with the outer surface of the catheter body 102. In other embodiments, the magnets 110A-C may be positioned radially within the catheter body 102 away from the outer surface of the catheter body 102. Each array of magnets 110A-C may be fixed in or on the catheter 100 by any suitable method. For example, in some variations the one or more arrays of magnets 110A-C may be embedded in, adhered to, or friction-fit within the catheter 100.

Still referring to FIG. 1, the catheter 100 may include one or more nesting regions 106A-E. One or more of the nesting regions, such as the nesting regions 106A and 106B may be positioned distally of the working site 150. One or more of the nesting regions, such as the nesting regions 106C, 106D, and 106E may be positioned proximally of the working site 150. Each of the one or more nesting regions 106A-E may be configured to geometrically engage with a nesting region 206A-E of the second catheter 200 (FIGS. 2 and 4), as discussed in greater detail below. Any or all of the nesting regions 106A-E may be magnetic. That is, in embodiments, and with specific reference to the nesting region 106A, the nesting region 106A may be, in its entirety, formed of one or more of the magnetic materials discussed above with respect to the magnetic arrays 110A-C. In other embodiments, and with specific reference to the nesting region 106A, the nesting region 106A may be partially formed of, or include, one or more of the magnetic materials discussed above with respect to the magnetic arrays 110A-C. Therefore, any or all of the nesting regions 106A-E may be configured to magnetically mate with a nesting region 206A-E of the second catheter 200 (FIGS. 2 and 4), as discussed in greater detail below. In other embodiments, the nesting regions 106A-E may be non-magnetic, that is made of a non-magnetic material.

Still referring to FIG. 1, the catheter 100 may include one or more ball bearings 112A-C. The one or more ball bearings 112A-C may be positioned along or within the catheter body 102. The one or more ball bearings 112A-C may be positioned between any components of the catheter 100 or portions of the catheter body 102 to increase the flexibility of the catheter 100. That is, the one or more ball bearings 112A-C may be positioned between any or all of the nesting regions 106A-E, the magnetic arrays 110A-C, and the working site 150. The one or more ball bearings 112A-C may generally be any rounded component that promotes flexion between the portions of the catheter body 102 positioned proximally and distally to the one or more ball bearings 112A-C.

Specific reference will now be made to the positioning of the nesting regions 106A-E along the catheter body 102. A nesting region, such as the nesting region 106A may be positioned proximally adjacent the distal tip 103 of the catheter 100.

In embodiments, a nesting region, such as the nesting region 106B, may be adjacent a magnetic array (e.g. the magnetic array 110A) at a first longitudinal end of the nesting region 106B and adjacent a ball bearing (e.g. the ball bearing 112A) at a second longitudinal end of the nesting region 106B. In embodiments, a nesting region, such as the nesting region 106D, may be adjacent a first magnetic array (e.g. the magnetic array 110B) at a first longitudinal end of the nesting region 106D and adjacent a second magnetic array (e.g. the magnetic array 110C) at a second longitudinal end of the nesting region 106D. In embodiments, a nesting region, such as the nesting region 106E, may be adjacent a first ball bearing (e.g. the ball bearing 112B) at a first longitudinal end of the nesting region 106E and adjacent a second ball bearing (e.g. the ball bearing 112C) at a second longitudinal end of the nesting region 106E.

In embodiments, a nesting region, such as the nesting region 106B, may be separated from the working site 150 by a ball bearing (e.g. the ball bearing 112A). In other words, a ball bearing, such as the ball bearing 112A, may be adjacent a nesting region (e.g. the nesting region 106B) at a first longitudinal end of the ball bearing 112A and adjacent the working site 150 at a second longitudinal end of the ball bearing 112A. In embodiments, a nesting region, such as the nesting region 106C, may be adjacent the working site 150.

With reference now to FIG. 3, which depicts a longitudinal cross section of the catheter 100, any or all of the nesting regions 106A-E may include a channel 108 extending therethrough. In embodiments, each of the nesting regions proximal to the working site 150, such as the nesting regions 106C, 106D, and 106E may include a channel 108C, 108D, 108E, respectively. The electrode wire 105 may extend proximally from the electrode 104 and through the catheter body 102 through each of the channels 108C, 108D, and 108E. It should be appreciated that in embodiments where the catheter 100 includes a modification device besides the electrode 104, that any elements associated with the modification device (e.g., conductive wires, mechanical pull wires, etc.) may extend through the channels of the nesting regions proximal to the working site 150 (i.e. the channels 108C, 108D, and 108E) to a proximal end of the catheter 100. It should further be appreciated any or all of the magnetic arrays 110A-C and/or any or all of the ball bearings 112A-C may include channels or hollow portions to allow for passage of elements therethrough.

Referring to FIGS. 1 and 3, any or all of the nesting regions 106A-E generally may include a groove body 140A-E and a groove recess 142A-E in each respective groove body 140A-E. With specific reference to the nesting region 106A, the groove recess 142A may generally be any recess, divot, indentation, cut-out, or the like in a surface of the groove body 140A. That is, the groove recess 142A extends from a first exterior surface of the groove body 140A, partially through an interior of the groove body 140A, toward a second exterior surface of the groove body 140A, and is void of material. Particularly, the groove recess 142A may be positioned along the active side 152 of the catheter 100, and therefore the active side 152 of the groove body 140A, and extend radially inward toward the non-active side 154 of the catheter 100 and groove body 140A.

In embodiments, a maximum height (e.g. in the direction of the z-axis of the coordinate axes of FIGS. 1 and 3) of the nesting region 106A may be equal to the height of the catheter body 102 (e.g. in the direction of the z-axis of the coordinate axes of FIGS. 1 and 3). The maximum height of the nesting region 106A may be defined by the height of the groove body 140A in an area of the groove body 140A that is not reduced by the groove recess 142A.

With specific reference to the nesting region 106C, the groove recess 142C may not extend into the channel 108C. That is, the channel 108C and groove recess 142C may be sized such that the interior of the channel 108C remains un-exposed to the groove recess 142C and vice versa. Therefore, the electrode wire 105 and/or any other components extending through the channel 108C may be prevented from entering the space of the groove recess 142C.

A nesting region, such as the nesting regions 106A-E, including a groove body (e.g. 140A-E) and groove recess (e.g. 142A-E) may generally be referred to herein as “a groove.”

Referring now to FIGS. 2 and 4, a catheter 200 (e.g. the second catheter of the system for forming a fistula) is depicted. The catheter 200 may resemble the catheter 100 discussed in FIGS. 1 and 3 in all aspects as except as discussed herein. That is, like components of the catheter 200 may mirror those of the catheter 100 unless specified. For instance, similar to the catheter 100, the catheter 200 may include a tip 203 and a catheter body 202 defining the working site 250. An electrode 204 or other modification device extending from the working site 250 may define the active side 252 diametrically opposite the non-active side 254. The catheter 200 may include one or more arrays of magnets 210A-C, one or more ball bearings 212A-C, and one or more nesting regions 206A-E. Any or all of the nesting regions 206A-E may include a channel (e.g. channels 208C, 208D, 208E) extending therethrough, which an electrode wire 205 may extend through.

Each of the one or more nesting regions 206A-E may be configured to geometrically engage with the nesting regions 106A-E of the first catheter 100, as discussed in greater detail below. Any or all of the nesting regions 206A-E may be magnetic. That is, in embodiments, and with specific reference to the nesting region 206A, the nesting region 206A may be, in its entirety, formed of one or more of the magnetic materials discussed above with respect to the magnetic arrays 110A-C. In other embodiments, and with specific reference to the nesting region 206A, the nesting region 206A may be partially formed of, or include, one or more of the magnetic materials discussed above with respect to the magnetic arrays 110A-C. Therefore, any or all of the nesting regions 206A-E may be configured to magnetically mate with the nesting regions 106A-E of the first catheter 100, as discussed in greater detail below. In some embodiments, the nesting regions 206A-E may be non-magnetic, that is, made of non-magnetic material.

Any or all of the nesting regions 206A-E generally may include a tongue body 240A-E and a tongue projection 243A-E that extends from each respective tongue body 240A-E. With specific reference to the nesting region 206A, the tongue projection 243A may generally be any projection, extension, protrusion, jut, or the like, from the tongue body 240A. That is, the tongue projection 243A extends from a first exterior surface of the tongue body 240A, radially outward from the tongue body 240A. The tongue projection 243A may be integral with and formed of the same material as the tongue body 240A. The tongue projection 243A may be positioned along the active side 252 of the catheter 200, and therefore the active side 252 of the tongue body 240A, and extend radially outward from the active side 252 of the catheter 200 and tongue body 240A.

In embodiments, a maximum height (e.g. in the direction of the z-axis of the coordinate axes of FIGS. 2 and 4) of the nesting region 206A may be greater than the height of catheter body 202 (e.g. in the direction of the z-axis of the coordinate axes of FIGS. 2 and 4). The maximum height of the nesting region 206A may be defined by the height of the tongue body 240A in addition to the maximum distance of radial extension of the tongue projection 243A from the tongue body 240A.

A nesting region, such as the nesting regions 206A-E, including a tongue body (e.g. 240A-E) and tongue projection (e.g. 243A-E) may generally be referred to herein as “a tongue.”

As noted above, the nesting regions 106A-E of the catheter 100 are configured to geometrically engage the nesting regions 206A-E of the catheter 200, and vice versa. Specific reference will now be made with respect to the nesting region 106A of the catheter 100 and the nesting region 206A of the catheter 200. The nesting region 106A of the catheter 100 and the nesting region 206A of the catheter 200 are configured to geometrically engage with each other. By being configured to geometrically engage, the nesting region 106A and the nesting region 206A are shaped and sized such that a surface of the nesting region 106A corresponds with a surface of the nesting region 206A, and vice versa. More particularly, the active side 252 of the nesting region 106A corresponds with the active side 252 of the nesting 206A, and vice versa. When geometrically engaged, at least a portion of the nesting region 206A is within at least a portion of the nesting region 106A. More specifically, when geometrically engaged, the tongue projection 243 A of the tongue body 240A of the nesting region 206A is received within the groove recess 142A of the groove body 140A of the nesting region 106A.

As noted above, the nesting region 106A and the nesting region 206A may each be at least partially formed of, or include, magnetic material. The magnetic material of the nesting region 106A may be configured to mate with the magnetic material of the nesting region 206A, and vice versa, such that the nesting region 106A and the nesting region 206A geometrically engage. As used herein, the term “mate” may be understood to mean a mutual attraction between a first magnet and a second magnet. The mutual attraction between the nesting region 106A and the nesting region 206A may be referred to herein as a lateral coupling force.

Referring now to FIGS. 1-6, a system and method for forming a fistula between the first blood vessel 300 and a second blood vessel 302 with the first catheter 100 and the second catheter 200 will now be discussed. While both the catheter 100 and the catheter 200 have been described herein as including an electrode 104, 204 extendable from the respective working sites 150, 250, of the catheters 100, 200, it should be appreciated that either of the first catheter 100 or the second catheter 200 may not include a modification device, such as the electrode 104, 204. For instance, and with specific reference to the first catheter 100 as shown in FIGS. 5 and 6, the active side 152 of the working site 150 may include a pocket 160 configured to receive the electrode 204 of the second catheter 200. The pocket 160 may be particularly shaped, sized, and/or the like to receive the electrode 204 of the second catheter 200 therein. In other embodiments, the second catheter 200 may include a recess in the active side 252 of the working site 250 to receive the electrode 104 of the first catheter 100.

Referring first to FIG. 5, the first catheter 100 may be advanced within a lumen of the blood vessel 300. The second catheter 200 may be placed in a blood vessel 302 that is adjacent to the blood vessel 300.

The nesting region 106A the first catheter 100 and the nesting region 206A of the second catheter 200 may be configured to promote rotational and axial alignment of the catheters 100 and 200. Proper axial and rotational alignment between catheters 100 and 200 may facilitate alignment of one or more fistula-forming elements, such as the working sites 150, 250 of the first and second catheters 100, 200, respectively. More specifically, proper axial and rotational alignment between the first catheter 100 and the second catheter 200 may facilitate alignment of the electrode 204 with the pocket 160.

In embodiments where the nesting region 106A is magnetic, the magnetic material of the nesting region 106A may be arranged such that the magnetic field generated by the nesting region 106A is stronger in the direction of the active side 152 of the working site 150 (e.g. in the −z direction of the coordinate axes of FIGS. 5 and 6) than in the direction of the non-active side 154 of the working site 150 (e.g. in the +z direction of the coordinate axes of FIGS. 5 and 6). Similarly, in embodiments where the nesting region 206A is magnetic, the magnetic material of the nesting region 206A may be arranged such that the magnetic field generated by the nesting region 206A is stronger in the direction of the active side 252 of the working site 250 (e.g. in the +z direction of the coordinate axes of FIGS. 5 and 6) than in the direction of the non-active side 254 of the working site 250 (e.g. in the −z direction of the coordinate axes of FIGS. 5 and 6). In such embodiments, the strength of the magnetic fields in the directions of the active sides 152, 252 of the working sites 150, 250, respectively, and polarity of the magnetic fields may promote rotational alignment between the active side 152 of the working site 150 of first catheter 100 in the first blood vessel 300 and the active side 252 of the working site 250 of the second catheter 200 in the second blood vessel 302. Moreover, the strength of the magnetic fields in the directions of the active sides 152, 252 of the catheters 100, 200, respectively, and polarity of the magnetic fields may promote rotational alignment between the groove recess 142A of the groove body 140A of the nesting region 106A and the tongue projection 243A of the tongue body 240A of the nesting region 206A.

For ease of illustration in FIGS. 5 and 6, the first catheter 100 and the second catheter 200 are not depicted as having the one or more arrays of magnets 110A-C and 210A-C, respectively. However, in embodiments where the first catheter 100 and the second catheter 200 include the one or more arrays of magnets 110A-C and 210A-C, respectively, the one or more arrays of magnets 110A-C of the first catheter 100 and the one or more arrays of magnets 210A-C of the second catheter 200 may be configured to promote rotational and axial alignment of the catheters 100 and 200, as described above. The one or more arrays of magnets 110A-C of the first catheter 100 may be arranged such that the magnetic fields generated by the one or more arrays of magnets 110A-C are stronger in the direction of the active side 152 of the working site 150 (e.g. in the −z direction of the coordinate axes of FIGS. 5 and 6) than in the direction of the non-active side 154 of the working site 150 (e.g. in the +z direction of the coordinate axes of FIGS. 5 and 6) and attract to the one or more arrays of magnets 210A-C of the second catheter 200 due to alignment of opposing N and S polarities. Similarly, the one or more arrays of magnets 210A-C of the second catheter 200 may be arranged such that the magnetic fields generated by the one or more arrays of magnets 210A-C are stronger in the direction of the active side 252 of the working site 250 (e.g. in the +z direction of the coordinate axes of FIGS. 5 and 6) than in the direction of the non-active side 254 of the working site 250 (e.g. in the −z direction of the coordinate axes of FIGS. 5 and 6) and attract to the one or more arrays of magnets 110A-C of the first catheter 100 due to alignment of opposing N and S polarities. Therefore, in such embodiments, the strength and polarity of the magnetic fields in the directions of the active sides 152, 252 of the working sites 150, 250, respectively, may promote rotational alignment between the active side 152 of the working site 150 of first catheter 100 in the first blood vessel 300 and the active side 252 of the working site 250 of the second catheter 200 in the second blood vessel 302. Moreover, the strength and polarity of the magnetic fields in the directions of the active sides 152, 252 of the catheters 100, 200, respectively, may promote rotational alignment between the groove recess 142A of the groove body 140A of the nesting region 106A and the tongue projection 243A of the tongue body 240A of the nesting region 206A.

The catheters 100 and 200, as depicted in FIG. 5 are axially misaligned, such that the electrode 204 of the second catheter 200 is not aligned with the pocket 160 of the first catheter 100 in the x-direction of the coordinate axes of FIGS. 5 and 6. Moreover, as depicted in FIG. 5, for instance, the first catheter 100 and the second catheter 200 are in weak coaptation. When in weak coaptation, space may remain between at least one of the active side 152 of the working site 150 of the first catheter 100 and an adjacent wall of the blood vessel 300 and the active side 252 of the working site 250 of the second catheter 200 and an adjacent wall of the blood vessel 302. Therefore, in weak coaptation, the active side 252 of the working site 250 and the active side 152 of the working site 150 are not in close approximation with one another (e.g. in the direction of the z-axis of the coordinate axes of FIGS. 5 and 6).

In embodiments, as depicted in FIGS. 1-4, the first catheter 100 and the second catheter may include multiple nesting regions 106A-E and 206A-E, respectively. In such embodiments, and where the nesting regions 106A-E and 206A-E are magnetic, each nesting region 106A-E of the first catheter 100 may be configured to mate with a corresponding nesting region 206A-E of the second catheter 200, and vice versa, such that the first catheter 100 and the second catheter 400 may be aligned and coapted. As used herein, the terms “coapted” and/or “strong coaptation” may be understood to mean that the first catheter 100 and the second catheter 200 are in close approximation (e.g. in the direction of the z-axis of the coordinate axes of FIGS. 5 and 6) such that the electrode 204 of the second catheter 200 may enter the pocket 160 of the first catheter 100. For instance, the nesting region 106A positioned a first distance distal (e.g. in the +x direction of the coordinate axes of FIGS. 1-4) of the working site 150 of the catheter 100 may be configured to mate with the nesting region 206A positioned the first distance distal (e.g. in the +x direction of the coordinate axes of FIGS. 1-4) the working site 250 of the second catheter 200. The nesting region 106B positioned a second distance distal (e.g. in the +x direction of the coordinate axes of FIGS. 1-4) the working site 150 of the catheter 100 may be configured to mate with the nesting region 206B positioned the second distance distal (e.g. in the +x direction of the coordinate axes of FIGS. 1-4) the working site 250 of the second catheter 200. Similarly, the nesting region 106C positioned a first distance proximal (e.g. in the −x direction of the coordinate axes of FIGS. 1-4) the working site 150 of the first catheter 100 may be configured to mate with the nesting region 206C positioned the first distance proximal (e.g. in the −x direction of the coordinate axes of FIGS. 1-4) the working site 250 of the second catheter 200, and so on.

Similarly, in embodiments where the first catheter 100 and the second catheter 200 include the one or more arrays of magnets 110A-C and 210A-C, respectively, each array of magnets 110A-C of the first catheter 100 may be configured to mate with a corresponding array of magnets 210A-C of the second catheter 200, and vice versa, such that the first catheter 100 and the second catheter 200 may be aligned and coapted. For instance, the array of magnets 110A positioned a first distance distal (e.g. in the +x direction of the coordinate axes of FIGS. 1-4) the working site 150 of the first catheter 100 may be configured to mate with the array of magnets 210A positioned the first distance distal (e.g. in the +x direction of the coordinate axes of FIGS. 1-4) the working site 250 of the second catheter 200. The array of magnets 110B positioned a first distance proximal (e.g. in the −x direction of the coordinate axes of FIGS. 1-4) the working site 150 of the first catheter 100 may be configured to mate with the array of magnets 210B positioned the first distance proximal (e.g. in the −x direction of the coordinate axes of FIGS. 1-4) the working site 250 of the second catheter 200. Similarly, the array of magnets 110C positioned a second distance proximal (e.g. in the −x direction of the coordinate axes of FIGS. 1-6) the working site 150 of the first catheter 100 may be configured to mate with the array of magnets 210C positioned the second distance proximal (e.g. in the −x direction of the coordinate axes of FIGS. 1-6) the working site 250 of the second catheter 200.

Referring now to FIG. 6, when the first catheter 100 and the second catheter 200 are aligned and coapted, the nesting region 106A of the first catheter 100 and the nesting region 206A of the second catheter 200 geometrically engage. In other words, the tongue projection 243A of the tongue body 240A of the nesting region 206A is received within the groove recess 142A of the groove body 140A of the nesting region 106A. As shown, the wall of the blood vessel 300 and the wall of the blood vessel 302 positioned between the nesting region 106A and the nesting 206A may deform according to the size and shape of the of the groove recess 142A and/or tongue projection 243A to allow the tongue projection 243A to be at least partially received within the groove recess 142A. When geometrically engaged, the nesting region 106A and the nesting region 206A, and more particularly the tongue projection 243A and groove recess 142A interlock. When interlocked, axial movement (e.g. in the direction of the x-axis of the coordinate axes of FIG. 6) of the nesting region 106A and the nesting region 206A relative to each other, and therefore of the catheters 100 and 200 relative to each other, is limited. When interlocked, a user must apply a greater axial force to the catheter 100 and/or 200 to disengage the nesting region 106A and the nesting region 206A, and more particularly the tongue projection 243A and the groove recess 142A, than required to move catheter 100 and/or the catheter 200 axially within the blood vessels 300, 302, respectively, when the nesting region 106A and the nesting region 206A are not geometrically engaged. By interlocking then, the nesting region 106A and the nesting region 206A promote the maintenance of the alignment and coaptation of the first catheter 100 and the second catheter 200. More particularly, by interlocking, the nesting region 106A and the nesting region 206A promote the maintenance of the alignment and coaptation of the working site 150 of first catheter 100 and the working site 250 of the second catheter 200.

Moreover, when the nesting region 106A of the first catheter 100 and the nesting region 206A of the second catheter 200 geometrically engage, tactile feedback may be sent through the first catheter 100 and/or the second catheter 200 to alert a user manipulating the handle of the first catheter 100 and/or the second catheter 200 that the first catheter 100 and second catheter 200 are aligned and coapted. For instance, as a user advances the first catheter 100 and the second catheter 200 within the blood vessels 300, 302, respectively, the catheter 100 and/or the catheter 200 may “catch” when the nesting region 106A geometrically engages the nesting region 206A. More particularly, when applying an axial force previously sufficient to advance the catheters 100, 200 in the blood vessels 300, 302, respectively, the user may feel the catheter 100 and/or the catheter 200 slow or halt when the nesting region 106A of the first catheter 100 interlocks with the nesting region 206A of the second catheter. The tactile feedback sent through the first catheter 100 and/or the second catheter 200 when the nesting region 206A and the nesting region 106A geometrically engage indicates to a user that the first catheter 100 and the second catheter 200 are aligned and coapted. Similarly, in embodiments, where the nesting regions 106A and 206A are magnetic, the user may feel the lateral coupling force acting on the catheter 100 and/or the catheter 200 at the respective handles of the catheter 100 and/or the catheter 200. That is, a user may feel a sudden force on the first catheter 100 in the −z direction of the coordinate axes of FIG. 6 and/or a sudden force on the second catheter 200 in the +z direction of the coordinate axes of FIG. 6 when the nesting region 106A of the first catheter 100 and the nesting region 206A of the second catheter mate and geometrically engage, indicating to the user that the first catheter 100 and the second catheter are aligned and coapted. After the first catheter 100 and the second catheter 200 are coapted and aligned, the electrode 204 may be advanced and energized to ablate the wall of the second blood vessel 302 and the wall of the first blood vessel 300 and advanced into the pocket 160 of the first catheter 100, thereby forming a fistula between the first and second blood vessels 300, 302.

To further aid a user in determining when the first catheter 100 and/or second catheter are aligned and coapted, the nesting region 106A and the nesting region 206A may be made of a material that exhibits high radiopacity, allowing the nesting regions 106A, 206A to be visualized under fluoroscopy. By visually analyzing the catheters under fluoroscopy, a user can determine that the nesting region 106A and the nesting region 206A are geometrically engaged, thereby indicating that the working sites 150, 250 of the first catheter 100 and second catheter 200, respectively, are also aligned and coapted.

In embodiments where the first catheter 100 and the second catheter 200 include multiple nesting regions 106A-E and 206A-E, respectively, each nesting region 106A-E of the first catheter 100 may be configured to geometrically engage with a corresponding nesting region 206A-E of the second catheter 200, and vice versa, such that the first catheter 100 and the second catheter 200 may be aligned and coapted at least in part by the geometric engagements between the nesting regions 106A-E and 206A-E. For instance, the nesting region 106A positioned a first distance distal (e.g. in the +x direction of the coordinate axes of FIGS. 1-4) of the working site 150 of the catheter 100 may include a groove recess 142A of a first dimension, and the nesting region 206A positioned the first distance distal (e.g. in the +x direction of the coordinate axes of FIGS. 1-4) the working site 250 of the second catheter 200 may include a tongue projection 243A of a corresponding first dimension. The nesting region 106B positioned a second distance distal (e.g. in the +x direction of the coordinate axes of FIGS. 1-4) the working site 150 of the catheter 100 may include a groove recess 142B of a second dimension, and the nesting region 206B positioned the second distance distal (e.g. in the +x direction of the coordinate axes of FIGS. 1-4) may include a tongue projection 243B of a corresponding second dimension. In other words, the groove recess 142A of the nesting region 106A and the tongue projection 243A of the nesting region 206A may be sized and shaped such that the nesting regions 106A and 206A may only geometrically engage with each other. More generally, each groove recess of the first catheter 100 may be configured to geometrically engage with only one of the tongue projections of the second catheter 200, and in particular only with the tongue projection which aligns with each groove recess when the working sites 150 and 250 are aligned. Therefore, the groove recess 142A of the nesting region 106A and the tongue projection 243B of the nesting region 240B may be sized and shaped such that the nesting regions 106A and 206B do not geometrically engage with each other, and the groove recess 142B of the nesting region 106B and the tongue projection 243A of the nesting region 206A may be sized and shaped such that the nesting regions 106B and 206A do not geometrically engage with each other. Moreover, the groove recess 142B of the nesting region 106B and the tongue projection 243B of the nesting region 206B may be sized and shaped such that the nesting regions 106B and 206B may only geometrically engage with each other.

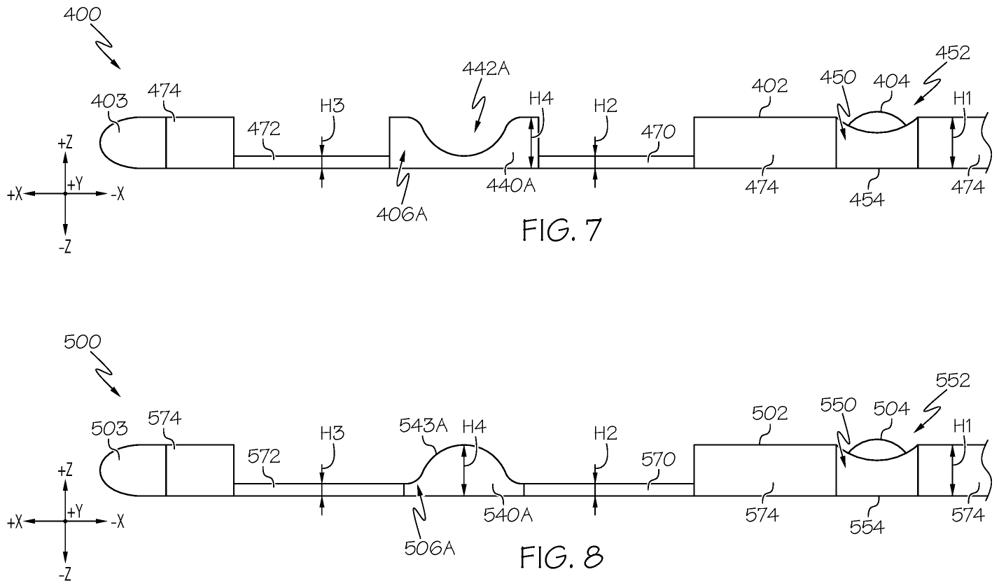

Referring now to FIG. 7, a catheter 400 is depicted. The catheter 400 may resemble the catheter 100 previously discussed in all aspects except as noted herein. That is, like components of the catheter 400 may mirror those of the catheter 100 unless specified. For instance, similar to the catheter 100, the catheter 400 may include a tip 403 and a catheter body 402 defining the working site 450. An electrode 404 or other modification device extending from the working site 450 may define the active side 452 diametrically opposite the non-active side 454. Although not depicted, it should be appreciated that the catheter 400 may include any or all of the arrays of magnets 110A-C (FIG. 1) and ball bearings 112A-C (FIG. 1). The catheter further includes a nesting region 406A having a groove body 440A and a groove recess 442A. The nesting region 406A may be magnetic.

The catheter body 402 includes a first portion 474 having a first height H1 (e.g. in the direction of z-axis of the coordinate axes of FIG. 7). The first height H1 of the first portion 474 of the catheter body 402 may be a maximum height of the catheter body 402. The catheter body 402 further includes a first transition region 470 positioned between the first portion 474 of the catheter body 402 and the nesting region 406A. The catheter body 402 may further include a second transition region 472 positioned between the first portion 474 and the nesting region 406A opposite the first transition region 470. That is, the first transition region 470 may be proximally adjacent the nesting region 406A, and the second transition region 472 may be distally adjacent the nesting region 406A. The first transition region 470 may have a second height H2, and the second transition region 472 may have a third height H3. The second height H2 and the third height H3 may both be less than the first height H1 of the first portion 474 of the catheter body 402. In embodiments, the second height H2 of the first transition region 470 and the third height H3 of the second transition region 472 may be equal. The nesting region 406A may have a maximum height H4 equal to or less than the first height H1 of the first portion 474 of the catheter body 402.

Referring now to FIG. 8, a catheter 500 is depicted. The catheter 500 may resemble the catheter 400 previously discussed in all aspects except as noted herein. For instance, similar to the catheter 400, the catheter 500 may include a tip 503 and a catheter body 502 defining the working site 550. An electrode 504 or other modification device extending from the working site 550 may define the active side 552 diametrically opposite the non-active side 554. Although not depicted, it should be appreciated that the catheter 500 may include any or all of the arrays of magnets 110A-C (FIG. 1) and ball bearings 112A-C (FIG. 1). The catheter further includes a nesting region 506A having a tongue body 540A and a tongue projection 543A. The nesting region 506A may be magnetic.

The catheter body 502 includes a first portion 574 having a first height H1 (e.g. in the direction of z-axis of the coordinate axes of FIG. 8). The first height H1 of the first portion 574 of the catheter body 502 may be a maximum height of the catheter body 502. The catheter body 502 further includes a first transition region 570 positioned between the first portion 574 of the catheter body 502 and the nesting region 506A. The catheter body 502 may further include a second transition region 572 positioned between the first portion 574 and the nesting region 506A opposite the first transition region 570. That is, the first transition region 570 may be proximally adjacent the nesting region 506A, and the second transition region 572 may be distally adjacent the nesting region 506A. The first transition region 570 may have a second height H2, and the second transition region 572 may have a third height H3. The second height H2 and the third height H3 may both be less than the first height H1 of the first portion 574 of the catheter body 502. In embodiments, the second height H2 of the first transition region 570 and the third height H3 of the second transition region 572 may be equal. The nesting region 506A may have a maximum height H4 equal to or less than the first height H1 of the first portion 574 of the catheter body 502.

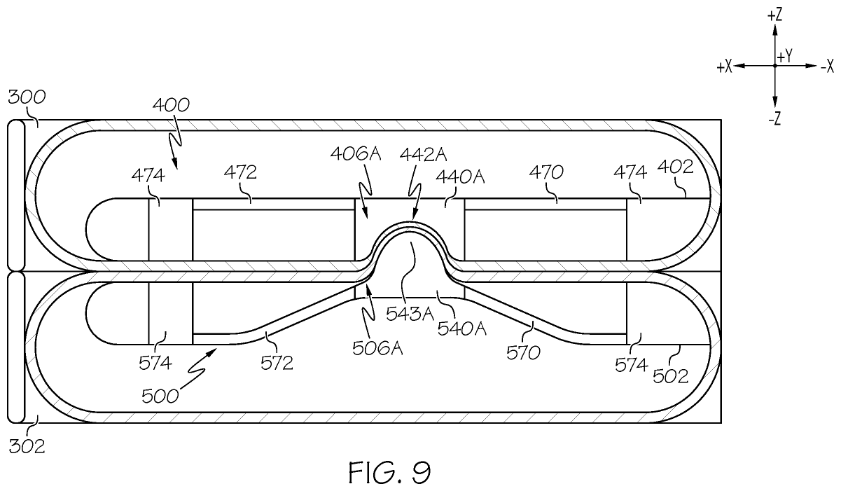

In embodiments incorporating the first transition regions 470, 570 and second transition regions 472, 572, the heights or profiles of the catheters 100, 200 may be maintained or reduced. That is, the nesting region 406A and/or the nesting region 506A need not have a maximum height greater than the maximum heights of the catheter bodies 402, 502 to geometrically engage with each other. Referring to FIG. 9, the reduced height of the transition regions 470, 472 of the catheter 400 may increase the flexibility of the transition regions 470, 472 compared to the first portion 474 of the catheter body 402. Similarly, the reduced height of the transition regions 570, 572 of the catheter 500 may increase the flexibility of the transition regions 570, 572 compared to the first portion 574 of the catheter 500. Therefore, as shown in FIG. 9, the increased flexibilities of the transition regions 470, 472 and/or the transition regions 570, 572, allow the nesting region 506A of the catheter 500 and the nesting region 406A of the catheter 400 to geometrically engage as a result of the magnetic mating forces between the nesting region 506A and the nesting region 406A, for instance. While FIG. 9 depicts the catheters 400 and 500 being used with each other in a system to form a fistula, it should be appreciated that the catheter 500 may similarly align and coapt with the catheter 100 (FIG. 1), for instance, and the catheter 400 may similarly align and coapt with the catheter 200 (FIG. 2), for instance.

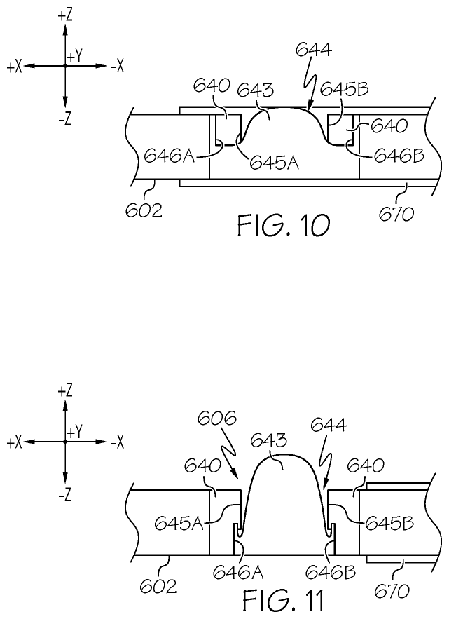

Referring now to FIGS. 10 and 11, a longitudinal cross section of a nesting region 606 is depicted. The nesting region 606 may be positioned along any catheter body of the above-described catheters. The nesting region 606 includes a tongue body 640 and a tongue projection 643. The tongue body 640 may include an opening 644 in the active side of the tongue body 640. The tongue body 640 may include a first ledge 645A and a second ledge 645B that extend from the opening 644 toward the non-active side of the tongue body 640. The tongue projection 643 may include a first flange 646A and a second flange 646B.

In some embodiments, the tongue projection 643 may expand from a low-profile configuration, depicted in FIG. 10, to an extended configuration, depicted in FIG. 11. In the low-profile configuration, the tongue projection 643 may be positioned in a non-engaging state, in which the tongue projection 643 may not be able to geometrically engage with a groove recess 142A of a corresponding nesting region 106A (FIG. 1). In the low-profile configuration, the tongue projection 643 may be maintained substantially flush with or within the tongue body 640 of the nesting region 606. In the low-profile configuration, the tongue projection 643 may also be maintained substantially flush with or within a catheter body 602. In the extended configuration, at least a portion of the tongue projection 643 may radially or laterally extend outward from the outer surface of the tongue body 640 to be in an engaging state, in which at least a portion of the tongue projection 643 is positioned to geometrically engage a groove recess 142A of a corresponding nesting region 106A (FIG. 1). Accordingly, a maximum distance of radial deflection of the tongue projection 643 from the outer surface of the tongue body 640, when in the extended configuration, may be greater than the maximum distance of radial deflection of the tongue projection 643 from the outer surface of the tongue body 640, when in the low-profile configuration.

In embodiments, the tongue projection 643 described herein may be biased toward the extended configuration. That is, the tongue projection 643 may be configured to self-expand from the low-profile configuration to the extended configuration. Put yet another way, the tongue projection 643 may be in its natural resting state in the extended configuration. In embodiments, the tongue projection 643 may be made of a shape-memory alloy, such as copper-aluminum-nickel and/or nickel-titanium. In embodiments, a force may be required to hold the tongue projection 643 in the low-profile configuration. For instance, referring to FIGS. 10 and 11, a sheath 670 may be advanced distally (e.g. in the +x direction of the coordinate axes of FIGS. 10 and 11) to maintain the tongue projection 643 in the low-profile configuration. The sheath 670 may include an inner lumen having a greater diameter than the catheter body 602 and the nesting region 606, thereby allowing the sheath 670 to be advanced distally over the catheter body 602 and nesting region 606, as depicted in FIG. 10. Thus, with the sheath 670 advanced distally over the catheter body 602, the tongue projection 643 may be compressed by the sheath 670 and maintained in the low-profile configuration. Retraction of the sheath 670 in the proximal direction (e.g. in the −x direction of the coordinate axes of FIGS. 10 and 11), exposes the tongue projection 643. With the sheath 670 no longer applying a force to the tongue projection 643 to maintain the tongue projection 643 in the low-profile configuration, and due to the natural bias of the tongue projection 643, the tongue projection 643 may naturally expand into the extended configuration, as shown in FIG. 11.

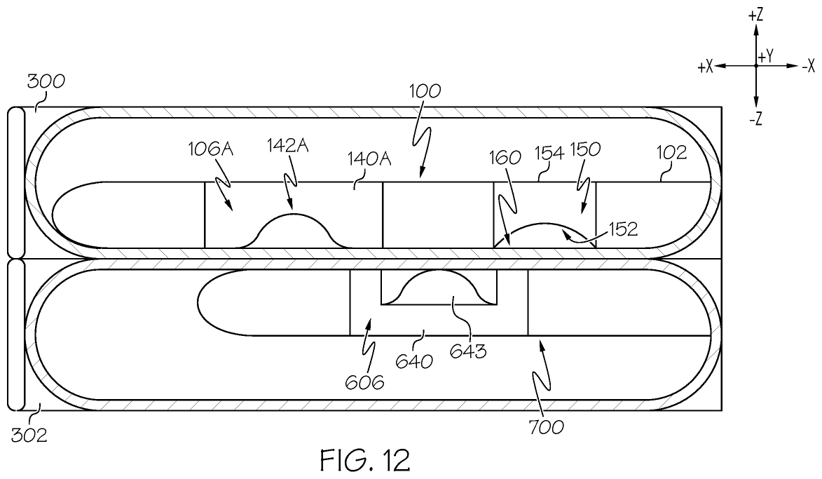

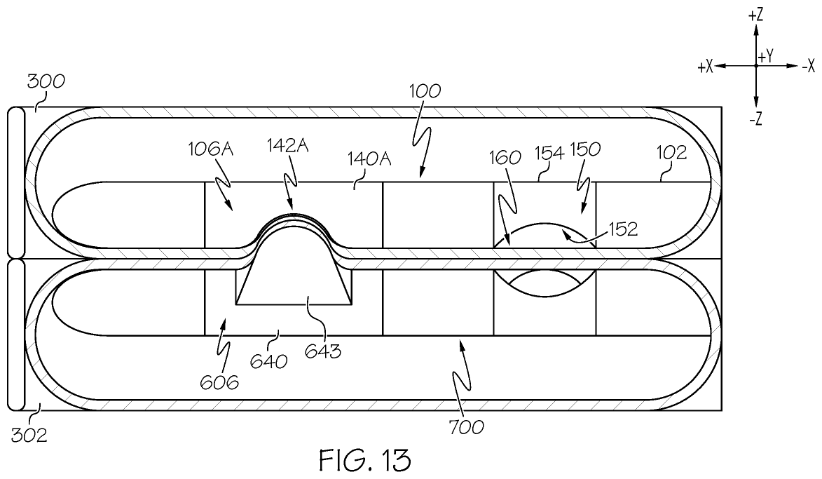

In some embodiments, the tongue projection 643 may be maintained in the low-profile configuration without the sheath 670. For instance, referring to FIGS. 12 and 13, a system for forming a fistula including a catheter 700 including the nesting region 606 is depicted. The tongue projection 643 may be maintained in the low-profile configuration by reaction forces exhibited on the tongue projection 643 by the wall of the blood vessel 302, wall of the blood vessel 300, and catheter 100 positioned in the blood vessel 300. For instance, as the catheter 700 is advanced in the vessel 300, the portions of the catheter body 102 of the catheter 100 not including the nesting region 106A may exhibit a reaction force against the natural bias of the tongue projection 643 to maintain the tongue projection 643 in the low-profile configuration. After the nesting region 606 of the catheter 700 is aligned with the nesting region 106A of the catheter 100, including the groove recess 142A correspondingly sized to receive the tongue projection 643, the tongue projection 643 may transition to the extended configuration. In other words, the groove recess 142A may not exhibit a reaction force against the tongue projection 643, thereby allowing the tongue projection 643 to transition to the extended configuration and geometrically engage with the groove recess 142A.

Referring back to FIGS. 10 and 11, the ledges 645A, 645B of the tongue body 640 and the flanges 646A, 646B of the tongue projection 643 may be sized such that when the tongue projection 643 transitions into the extended configuration, the flanges 646A, 646B catch on the ledges 645A, 645B of the tongue body 640. The interaction between the ledges 645A, 645B of the tongue body 640 and the flanges 646A, 646B of the tongue projection 643 prevent the flanges 646A, 646B of the tongue projection 643 from passing out of the opening 644 of the tongue body 640. Therefore, the ledges 645A, 645B of the tongue body 640 and the flanges 646A, 646B of the tongue projection 643 maintain the tongue projection 643 as part of the nesting region 606. It should be appreciated that incorporation of the ledges 645A, 645B of the tongue body 640 and the flanges 646A, 646B of the tongue projection 643 is merely an example of one method for maintaining the tongue projection 643 as part of the nesting region 606 when the tongue projection transitions into the extended configuration. For instance, in other embodiments, one or more tracks may be positioned along the interior of the non-active side of the tongue body 640, and the tongue projection 643 may be slidably tethered to the track by means of an anchor member. The anchor member may be sized to slide within, but not exit or detach from, the track.

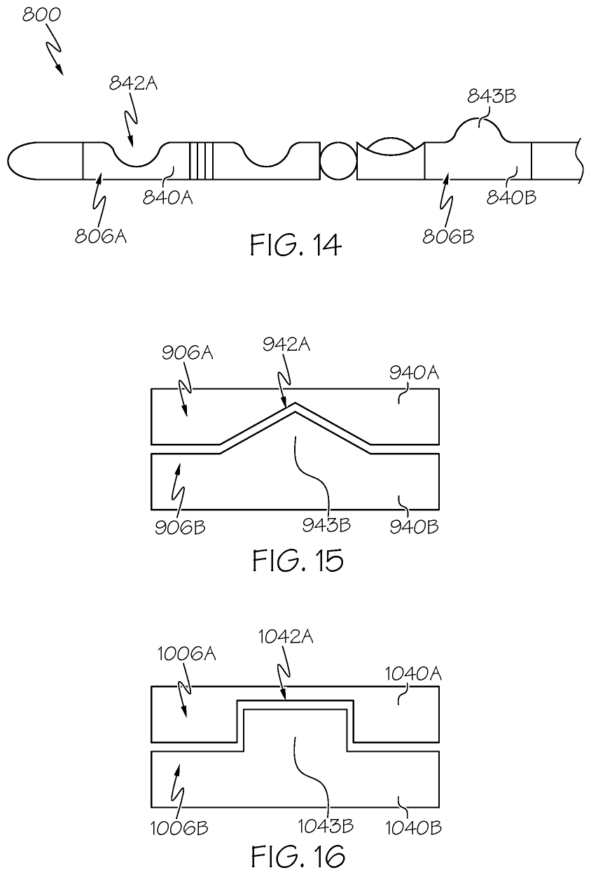

Embodiments have so far been depicted and discussed where a catheter, such as the catheter 100 (FIG. 1), includes nesting regions 106A-E (FIG. 1) of a first type. That is, each of the nesting regions 106A-E (FIG. 1) of the catheter 100 (FIG. 1) are grooves. Similarly, each of the nesting regions 206A-E (FIG. 2) of the catheter 200 (FIG. 2) are tongues. However, it should be appreciated that these are non-limiting examples. For example, and with reference to FIG. 14, a catheter 800 may include any combination of nesting regions. Specifically, the catheter 800 may include a nesting region 806A having a groove body 840A and a groove recess 842A. The catheter 800 may further include a nesting region 806B having a tongue body 840B and a tongue projection 843B. It should be appreciated that the catheter 800 may include any combination of types (i.e. groove and tongue) nesting regions, in both number and position along the catheter 800. It should be appreciated that a complimentary catheter to the catheter 800 in a system for forming fistula includes corresponding nesting regions configured to geometrically engage the nesting regions 806A and 806B of the catheter 800.

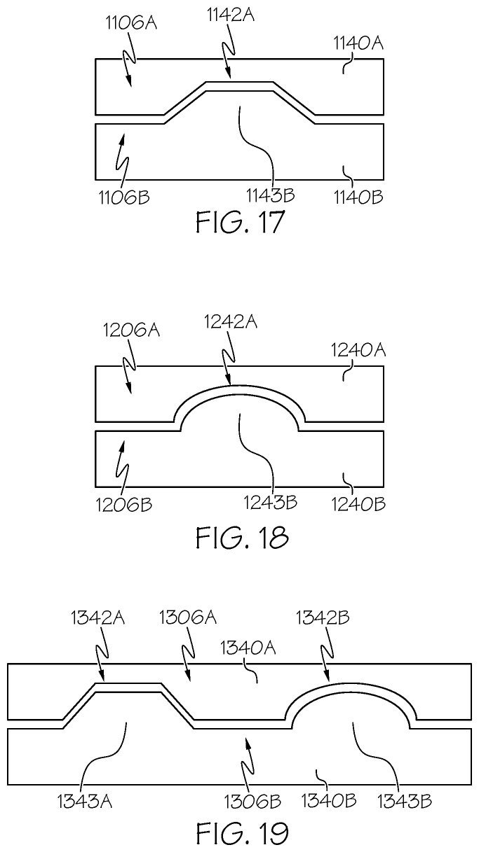

It should be appreciated that the nesting regions discussed herein can take any desirable shape. For instance, the nesting regions 106A-E (FIGS. 1) and 206A-E (FIG. 2) may have substantially parabolic groove recesses (142A-E) and tongue projections (243A-E), respectively. Referring to FIG. 15, a nesting region 906A may include a groove body 940A and a groove recess 942A having a substantially triangular shape. A complimentary nesting region 906B may include a tongue body 940B and a tongue projection 943B having a substantially triangular shape. Referring to FIG. 16, a nesting region 1006A may include a groove body 1040A and a groove recess 1042A having a substantially rectangular shape. A complimentary nesting region 1006B may include a tongue body 1040B and a tongue projection 1043B having a substantially rectangular shape. Referring to FIG. 17, a nesting region 1106A may include a groove body 1140A and a groove recess 1142A having a substantially trapezoidal shape. A complimentary nesting region 1106B may include a tongue body 1140B and a tongue projection 1143B having a substantially trapezoidal shape. Referring to FIG. 18, a nesting region 1206A may include a groove body 1240A and a groove recess 1242A having a substantially elliptical shape. A complimentary nesting region 1206B may include a tongue body 1240B and a tongue projection 1243B having a substantially elliptical shape. The particular shape and depth of the nesting regions may be chosen depending on the particular anatomy the catheters featuring the nesting regions will be employed in. Particularly, the shape and depth of the nesting regions may be chosen depending on the thickness, compressibility, and density of the vessel walls of the vessels 300, 302 (FIG. 5) the catheters featuring the nesting regions will be employed in. As noted above, when complimentary nesting regions are interlocked in the vessels 300, 302 (FIG. 5), the walls of the vessels 300, 302 (FIG. 5) are positioned between the nesting regions and may be at least partially deformed by the nesting regions. Therefore, the nesting regions may be shaped and sized to be as atraumatic as possible to the walls of the vessels 300, 302 (FIG. 5). For instance, any or all of the nesting regions depicted in FIGS. 15-18 may be modified such that any sharp angles are replaced with smooth, rounded transitions in the shapes of the nesting regions.

Referring now to FIG. 19, it should be appreciate that any of the above-discussed catheters may include nesting regions of different shapes, dimensions, and designs, as depicted. Moreover, it should be appreciated that a single nesting region, such as the nesting region 1306A, may include a single body for multiple nesting sites, which may be of different designs. For instance, the nesting region 1306A includes a groove body 1340A having a first groove recess 1342A and a second groove recess 1342B. The first groove recess 1342A may be substantially trapezoidal in shape, and the second groove recess 1342B may be substantially elliptical in shape. Similarly, the nesting region 1306B includes a tongue body 1340B having a first tongue projection 1343A and a second tongue projection 1343B. The first tongue projection 1343A may be substantially trapezoidal in shape, and the second tongue projection 1343B may be substantially elliptical in shape.

Embodiments can be described with reference to the following numerical clause:

1. A catheter, comprising: a catheter body, a modification device, and a nesting region, wherein: the modification device is configured to project from a working site of the catheter; and the nesting region is configured to geometrically engage a nesting region of a second catheter.

2. The catheter of clause 1, wherein: the modification device is an electrode; and the electrode comprises an electrode wire extending proximally through the catheter body from the working site.

3. The catheter of any preceding clause, wherein the nesting region is proximal the working site.

4. The catheter of any preceding clause, wherein: the nesting region comprises a channel; and the electrode wire extends through the channel.

5. The catheter of any preceding clause, wherein the nesting region is distal the working site.

6. The catheter of any preceding clause, wherein at least a portion of the nesting region is made of a magnetic material.

7. The catheter of any preceding clause, wherein the nesting region is configured to magnetically mate with the nesting region of the second catheter.

8. The catheter of any preceding clause, further comprising one or more arrays of magnets longitudinally positioned along the catheter body.

9 The catheter of any preceding clause, further comprising a ball bearing positioned adjacent the nesting region and between the nesting region and a portion of the catheter body.

10. The catheter of any preceding clause, wherein the catheter body comprises: a first portion having a first height; and a transition region having a second height, wherein: the second height is less than the first height; and the transition region is positioned between the first portion and the nesting region.

11. The catheter of any preceding clause, wherein a maximum height of the nesting region is equal to or less than the first height.

12. The catheter of any preceding clause, wherein: the catheter body has a first height; and a maximum height of the nesting region is a second height greater than the first height.

13. The catheter of any preceding clause, wherein the nesting region comprises a tongue body and a tongue projection that extends from the tongue body.

14. The catheter of any preceding clause, wherein the tongue projection is naturally spring biased from a low-profile configuration, wherein at least a portion of the tongue projection is positioned inside the catheter, to an extended position, wherein the portion of the tongue projection extends laterally away from the catheter body.

15. The catheter of any preceding clause, wherein: the modification device defines an active side of the catheter; and the tongue projection extends from the tongue body along the active side of the catheter.

16. The catheter of any preceding clause, wherein the nesting region of the second catheter comprises a recess configured to receive the tongue projection.

17. The catheter of any preceding clause, wherein the nesting region comprises a groove body and a groove recess in the groove body.

18. The catheter of any preceding clause, wherein: the modification device defines an active side of the catheter; and the groove recess is positioned along the active side of the catheter.

19. The catheter of any preceding clause, wherein the nesting region of the second catheter comprises a tongue projection configured to be received in the groove recess.

20. A system for forming a fistula between two blood vessels, comprising: a first catheter comprising a first catheter body and a tongue, wherein: the tongue comprises a tongue body and a tongue projection that extends from the tongue body; and a second catheter comprising a second catheter body and a groove, wherein: the groove comprises a groove body and a groove recess in the groove body; and the groove recess is configured to receive the tongue projection, wherein: at least one of the first catheter or the second catheter comprises a modification device.

21. The system of clause 20, wherein: the first catheter interlocks with the second catheter when the tongue projection is received in the groove recess.

22. The system of any preceding clause, wherein the first catheter is configured to be positioned within a first blood vessel, and the second catheter is configured to be positioned within a second blood vessel adjacent to the first blood vessel.

23. The system of any preceding clause, wherein a wall of the first blood vessel and a wall of the second blood vessel are positioned within the groove recess between the groove and the tongue projection when the tongue projection is received in the groove recess.

24. The system of any preceding clause, wherein at least a portion of the tongue and at least a portion of the groove are made of magnetic materials.

25. The system of any preceding clause, wherein the tongue and the groove are configured to magnetically mate.

26. The system of any preceding clause, wherein: the first catheter further comprises a working site; and the second catheter further comprises a working site, wherein: the fistula is formed between the working site of the first catheter and the working site of the second catheter; and the working site of the first catheter and the working site of the second catheter are aligned when the tongue projection is received in the groove recess.

27. A method of forming a fistula between a first blood vessel and a second blood vessel, comprising: advancing a first catheter into the first blood vessel, wherein the first catheter comprises: a first catheter body, a first working site, and a tongue, wherein: the tongue comprises a tongue body and a tongue projection that extends from the tongue body; advancing a second catheter into the second blood vessel adjacent to the first blood vessel, wherein the second catheter comprises: a second catheter body, a second working site, and a groove, wherein: the groove comprises a groove body and a groove recess in the groove body, wherein: the groove recess is configured to receive the tongue projection; and forming the fistula between the first blood vessel and the second blood vessel at the first working site and the second working site.

28. The method of clause 27, wherein: the tongue projection is received in the groove recess; and the first catheter interlocks with the second catheter when the tongue projection is received in the groove recess.

29. The method of any preceding clause, wherein: the tongue projection is received in the groove recess; and a wall of the first blood vessel and a wall of the second blood vessel are positioned within the groove recess between the groove and the tongue projection when the tongue projection is received in the groove recess.

30. The method of any preceding clause, wherein at least a portion of the tongue and at least a portion of the groove are magnetic.

31. The method of any preceding clause, wherein the tongue and the groove are configured to magnetically mate.

32. The method of any preceding clause, wherein: the tongue projection is received in the groove recess; and the working site of the first catheter and the working site of the second catheter are aligned when the tongue projection is received in the groove recess.

33. The method of any preceding clause, wherein: the first catheter comprises an electrode configured to project from the first working site; and the second catheter comprises a pocket in the second working site, wherein the pocket is configured to receive at least a portion of the electrode.

34. The method of any preceding clause, wherein: the second catheter comprises an electrode configured to project from the second working site; and the first catheter comprises a pocket in the first working site, wherein the pocket is configured to receive at least a portion of the electrode.

It should now be understood that embodiments of the present disclosure are directed to devices, systems, and methods for forming a fistula between two blood vessels. In particular, the devices and methods for forming a fistula described herein may include a first catheter, having a catheter body, a modification device, such as an electrode, and a nesting region. The modification device is configured to project from a working site of the catheter and modify a blood vessel wall. The nesting region is configured to geometrically engage a nesting region of a second catheter having a corresponding geometry. When geometrically engaged, the nesting regions of the catheters may interlock. The nesting regions may further be able to magnetically mate to enhance geometric engagement. When geometrically engaged, the nesting regions promote alignment and coaptation between the first and second catheters in adjacent blood vessels. Particularly, geometric engagement of the nesting regions may promote the alignment and coaptation of the working site of the first catheter and a working site of the second catheter before and during fistula formation.

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A catheter, comprising:
a catheter body, a modification device, and a nesting region, wherein:
the modification device is configured to project from a working site of the catheter; and
the nesting region is configured to geometrically engage a nesting region of a second catheter.

2. The catheter of claim 1, wherein:
the modification device is an electrode; and
the electrode comprises an electrode wire extending proximally through the catheter body from the working site.

3. The catheter of claim 2, wherein the nesting region is proximal the working site.

4. The catheter of claim 3, wherein:
the nesting region comprises a channel; and
the electrode wire extends through the channel.

5. The catheter of claim 1, wherein the nesting region is distal the working site.

6. The catheter of claim 1, wherein at least a portion of the nesting region is made of a magnetic material.

7. The catheter of claim 6, wherein the nesting region is configured to magnetically mate with the nesting region of the second catheter.

8. The catheter of claim 1, further comprising one or more arrays of magnets longitudinally positioned along the catheter body.

9. The catheter of claim 1, further comprising a ball bearing positioned adjacent the nesting region and between the nesting region and a portion of the catheter body.

10. The catheter of claim 1, wherein the catheter body comprises:
a first portion having a first height; and
a transition region having a second height, wherein:
the second height is less than the first height; and
the transition region is positioned between the first portion and the nesting region.

11. The catheter of claim 10, wherein a maximum height of the nesting region is equal to or less than the first height.

12. The catheter of claim 1, wherein:
the catheter body has a first height; and
a maximum height of the nesting region is a second height greater than the first height.

13. The catheter of claim 1, wherein the nesting region comprises a tongue body and a tongue projection that extends from the tongue body.

14. The catheter of claim 13, wherein the tongue projection is naturally spring biased from a low-profile configuration, wherein at least a portion of the tongue projection is positioned inside the catheter, to an extended position, wherein the portion of the tongue projection extends laterally away from the catheter body.

15. The catheter of claim 13, wherein:
the modification device defines an active side of the catheter;
and the tongue projection extends from the tongue body along the active side of the catheter.

16. The catheter of claim 13, wherein the nesting region of the second catheter comprises a recess configured to receive the tongue projection.

17. The catheter of claim 1, wherein the nesting region comprises a groove body and a groove recess in the groove body.

18. The catheter of claim 17, wherein:
the modification device defines an active side of the catheter;
and the groove recess is positioned along the active side of the catheter.

19. The catheter of claim 17, wherein the nesting region of the second catheter comprises a tongue projection configured to be received in the groove recess.

20. A system for forming a fistula between two blood vessels, comprising:
a first catheter comprising a first catheter body and a tongue, wherein:
the tongue comprises a tongue body and a tongue projection that extends from the tongue body; and
a second catheter comprising a second catheter body and a groove, wherein:
the groove comprises a groove body and a groove recess in the groove body; and
the groove recess is configured to receive the tongue projection, wherein:
at least one of the first catheter or the second catheter comprises a modification device.

21. The system of claim 20, wherein:
the first catheter interlocks with the second catheter when the tongue projection is received in the groove recess.

22. The system of claim 20, wherein the first catheter is configured to be positioned within a first blood vessel, and the second catheter is configured to be positioned within a second blood vessel adjacent to the first blood vessel.

23. The system of claim 22, wherein a wall of the first blood vessel and a wall of the second blood vessel are positioned within the groove recess between the groove and the tongue projection when the tongue projection is received in the groove recess.

24. The system of claim 20, wherein at least a portion of the tongue and at least a portion of the groove are made of magnetic materials.

25. The system of claim 24, wherein the tongue and the groove are configured to magnetically mate.

26. The system of claim 20, wherein:
the first catheter further comprises a working site; and
the second catheter further comprises a working site, wherein:
the fistula is formed between the working site of the first catheter and the working site of the second catheter; and
the working site of the first catheter and the working site of the second catheter are aligned when the tongue projection is received in the groove recess.

27-34. (canceled)
